(19) United States
(12) Patent Application Publication
(10) Pub. No.: US 2011/0004281 A1
(43) Pub. Date: Jan. 6, 2011

(54) IMPLANTABLE ANCHOR WITH LOCKING CAM

(76) Inventor: Robert E. Jones, McKinney, TX (US)

Correspondence Address: ST. JUDE MEDICAL NEUROMODULATION DIVISION, 6901 PRESTON ROAD, PLANO, TX, 75024, US

(21) Appl. No.: 12/829,762

(22) Filed: Jul. 2, 2010

Related U.S. Application Data
(60) Provisional application No. 61/222,969, filed on Jul. 3, 2009.

(51) Int. Cl. A61N 1/05

(52) U.S. Cl. 607/116

(57) ABSTRACT

There is disclosed various embodiments of an implantable anchor for anchoring a medical lead within a patient. The implantable anchor includes a body having at least one lumen for receiving a medical lead, a cam integrated with the body and rotatable to extend into the lumen for engaging the medical lead and inhibiting the movement of the lead with respect to the anchor. The cam may include a handle for facilitating the rotation of the cam. A needle could be connected to the handle to facilitate the securing of the anchor to a portion of the patient.

### CROSS-REFERENCE TO RELATED APPLICATIONS

This application claims the benefit of U.S. Provisional Application No. 61/222,969, filed Jul. 3, 2009, which is incorporated herein by reference.

### TECHNICAL HELD

The present application is generally related to an implantable anchor for anchoring an electrical stimulation lead, a drug infusion catheter, an electrical stimulation lead, or other catheter of an implantable medical device system.

### BACKGROUND

A number of implantable Medical devices have been commercially distributed that allow various medical agents to be controllably infused after implantation of the respective device within a patient. For example, implantable medical devices are used for the infusion of insulin, opiates, anti-spasmodic drugs, intrahepatic chemotherapy agents, and other therapeutic agents in a number of countries subject to the regulatory requirements of those countries.

There are a number of benefits to the use of implantable infusion devices. For example, when the therapeutic agent is delivered directly to the therapy site (for opiates and baclofen), the amount of the therapeutic agent that is needed is much lower. Side-effects are generally minimized. Also, the therapeutic effect can be significantly greater as compared to intravenous introduction of therapeutic agents (again, for opiates and baclofen). Furthermore, implantable infusion devices eliminate patient overdosing or underdosing due to patient error or limited patient capacity.

Implantable infusion devices typically include a central housing that includes a reservoir to hold the infusate, a septum to allow infusate to be introduced into the reservoir, an energy source to drive the infusate from the reservoir and through an outlet port, and various flow control elements. The central housing portion of the device is typically implanted in a suitable subcutaneous region with the septum positioned immediately below the skin of the patient to facilitate access to the reservoir for refilling purposes.

To deliver the infusate from the reservoir, a catheter is usually attached to the outlet port of the central housing to receive the infusate outflow. The distal end of the catheter is implanted within the patient adjacent to the appropriate therapy site (e.g., at a suitable intrathecal location to allow introduction of an infusate directly into the spinal fluid of the patient). Typically, some mechanism is employed to anchor the catheter so that infusate will continue to be delivered to the appropriate site such as sutures and/or anchoring structures.

Similar anchoring is also used in spinal cord stimulation (SCS) systems. In SCS systems, a pulse generator is typically implanted within a subcutaneous pocket within the patient. An electrical lead is also implanted within the patient. The proximal end of the electrical lead is electrically coupled (either directly or via one or more extensions) to the pulse generator to receive electrical pulses from the pulse generator. The distal end of the electrical lead is positioned with electrodes of the lead disposed within the epidural space of the patient to deliver the electrical pulses to the spinal neural tissue of the patient. The efficacy of the electrical stimulation in treating chronic pain of the patient depends upon applying the electrical pulses to the appropriate neural tissue. Accordingly, it is desired to retain the stimulation lead at a relatively fixed position over time. For that reason, the electrical lead is anchored so that migration of the electrical lead does not occur.

### SUMMARY

Ire one embodiment, there is disclosed various embodiments of an implantable anchor for anchoring a medical lead. In one embodiment, the implantable anchor may include a body having at least one lumen for receiving a medical lead. A cam is integrated with the body and rotatable between an open position and a closed position, and when in the close position, extends into the lumen for engaging the medical lead and inhibiting the movement of the lead with respect to the anchor. The cam may include a handle for facilitating the rotation of the cam. A needle could be connected to the handle to facilitate the securing of the anchor to a portion of the patient.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 is an isometric view illustrating one embodiment of an implantable anchor.

FIG. 2 is a side view illustrating one embodiment of an implantable anchor as similarly shown in FIG. 1.

FIG. 3 is a longitudinal cross-sectional view of the embodiment illustrated in FIGS. 1 and 2 illustrating the implantable anchor in a first or open configuration.

FIG. 4 is a longitudinal cross-sectional view of the embodiment illustrated in FIGS. 1 and 2 illustrating the implantable anchor second or closed configuration.

FIG. 5 is an isometric view illustrating an embodiment of an implantable anchor with two lumens.

FIG. 6 is an isometric view illustrating an embodiment of an implantable anchor with a locking arm.

FIG. 7 is a longitudinal cross-sectional view of the embodiment illustrated in FIG. 6 illustrating the implantable anchor in closed configuration.

FIG. 8 is an isometric view illustrating an embodiment of an implantable anchor with a locking arm and suture in a first or open configuration.

FIG. 9 is a longitudinal cross-sectional view of the embodiment illustrated in FIG. 8 in the first or open configuration.

FIG. 10 is an isometric view illustrating the embodiment of FIG. 8 in a second or closed configuration.

FIG. 11 is a longitudinal cross-sectional view of the embodiment illustrated in FIG. 8 in the second or closed configuration.

FIG. 12 depicts a conventional neurostimulation system that may utilize an anchor according to at least one representative embodiment.

FIG. 13 depicts a conventional drug pump system that may utilize an anchor according to at least one representative embodiment.

### DETAILED DESCRIPTION

For the purposes of promoting an understanding of the principles of the present invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

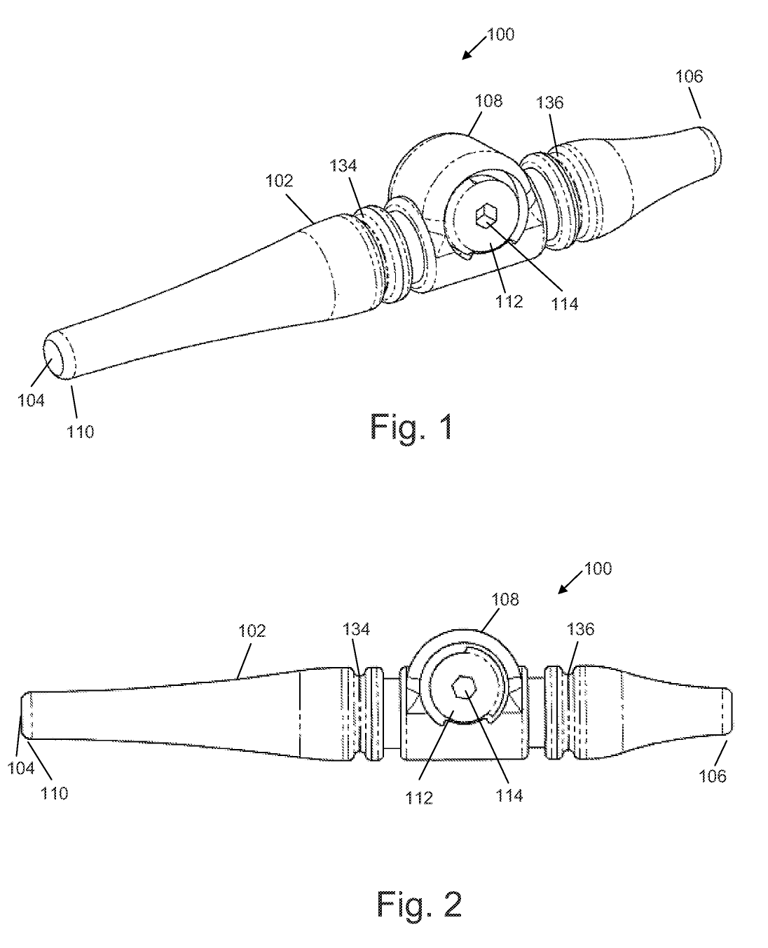

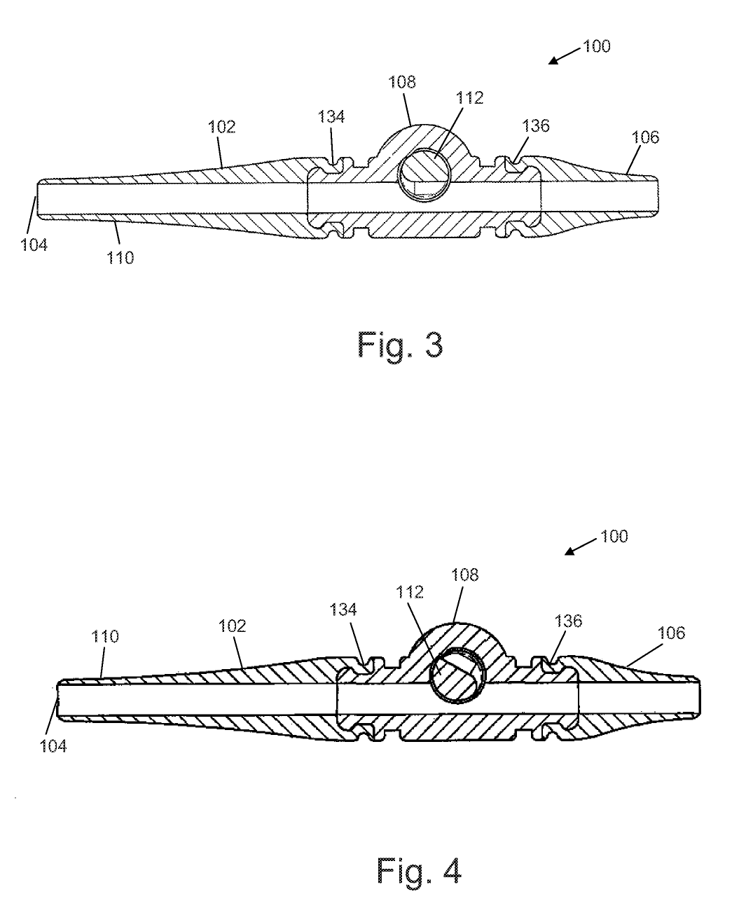

FIGS. 1 through 4 depict an implantable anchor 100, with FIG. 3 illustrating the anchor 100 in a first or open configuration and FIG. 4 illustrating the anchor 100 in a second or closed configuration. The anchor 100 may be used for anchoring a drug infusion catheter, an electrical lead, or other catheter (not shown) according to one representative embodiment. For the purpose of this disclosure, the term “lead” is used in a broad manner and should be interpreted to encompass both infusion catheters and stimulation leads.

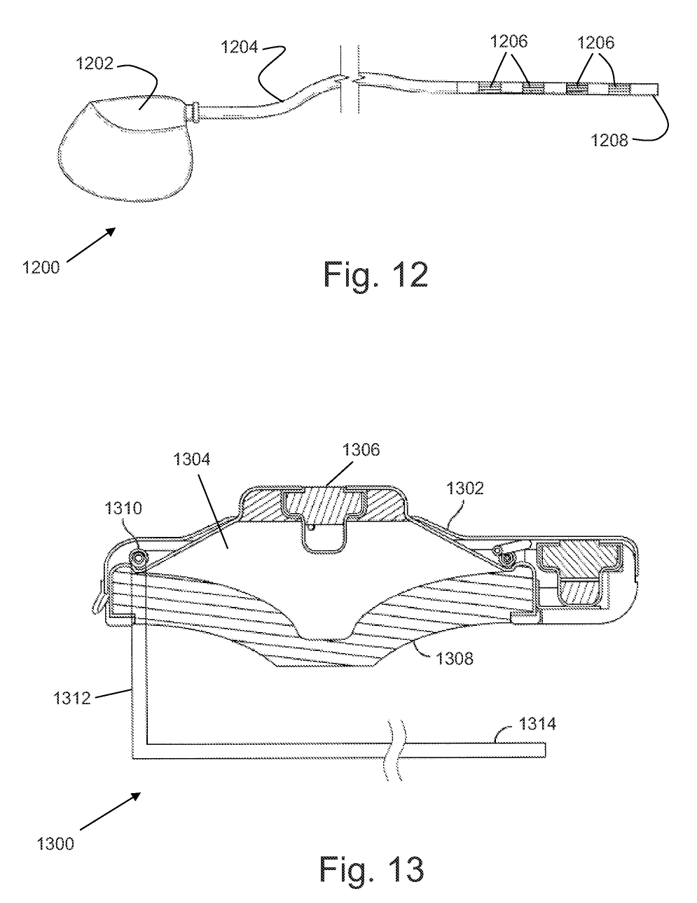

Anchor 100 includes a longitudinal body 102 with a lumen 104 defined therein. In the illustrative embodiment, lumen 104 runs longitudinally from end 106, through middle portion 108, to end 110, of the longitudinal body 102. As will be explained in greater detail below, the diameter of the longitudinal lumen may be sized accordingly to slidingly receive a lead of an SCS (see FIG. 11) or an implantable infusion system (see FIG. 12).

Middle portion 108 of anchor 100 includes a rotatable cam 112 that extends laterally through anchor 100. Cam 112 includes an interface 114 for receiving a tool to facilitate the axial rotation of cam 112 in anchor 100. Cam 112 is shaped such that, as depicted in FIG. 3, when cam 112 is positioned in a first or open position, cam 112 does not inhibit lumen 104 and thereby permits the insertion of a lead through lumen 104. As depicted in FIG. 4, cam 112, when rotated to a second or closed position, a portion of cam 112 extends into lumen 104, such that when a lead has been inserted through lumen 104, cam 112 engages the lead inhibiting the longitudinal and lateral movement of the lead with respect to anchor 100. It is contemplated that a locking mechanism, such as, but not limited to a ratcheting mechanism, be utilized to further lock cam 112 in the second or closed position and further inhibit the rotation of cam 112 from the second position back to the first position.

In use, cam 112 is place in the first or open position thereby permitting anchor 100 to be placed on the proximal end of a lead and slid over the lead until the anchor is properly positioned along the lead. Cam 112 is then rotated to the second or closed position, such that cam 112 engages the lead. Such engagement locks the anchor in place and inhibits movement of the anchor relative to the lead.

In certain embodiments, the anchor 100 may be fabricated using any suitable polymer processing technique. The polymer or polymers selected for the anchor 100 are preferably adapted for long term implantation. Biocompatibility and biostability are characteristics for the polymer selection for anchor 100. Also, the polymer preferably possesses a medium to high durometer to maintain the structural characteristics of anchor 100. An example of a suitable polymer for anchor 100 is polyetheretherketone (PEEK), although any biostable, biocompatible polymer having a suitable durometer and a suitable coefficient of friction can be employed.

In one embodiment, a combination of a relatively hard and soft (or flexible) material may be utilized. In this embodiment, the middle portion 108 of body 102 may be made from a relatively hard material, such as, but not limited to, select metals or PEEK. The end portions 106 and 110 may be fabricated from a more compliant material with a lower durometer value, such as silicone. Thus, the second end portions 106 and 110 are relatively flexible when compared to the middle portion 108 and may act as strain relief ports with respect to the middle portion 108.

In other embodiments, the longitudinal body 102 may be formed from a single piece of material. In such embodiments, the end portions may still function as strain relief ports. However, in these embodiments, the relative flexibility of the end portions may be due to geometric properties (such as thickness of the walls, etc.) and not from the different material properties of the component portions. Thus, the term flexibility as used herein may mean deformable (whether by choice of materials or geometry).

In some embodiments, the body 102 includes channels 134 and 136 which circumscribe body 102. Channels 134 and 136 are used to facilitate the suturing of anchor 100 to tissue of the patient.

The diameter of lumen 104 is sufficiently large to permit the introduction of a lead with little difficulty. Thus, when the cam 112 is in the open position (FIG. 3), the anchor 100 may be freely moved along the leads (not shown). However, when the cam 112 is rotated to the second or closed position (FIG. 4), a portion of cam 112 extends into lumen 104, thereby decreasing the corresponding size of portion lumen 104 smaller than the diameters of the leads. Thereby, upon rotation of cam 112 an open to a closed position, a compressive force is applied to the lead to cause anchor 100 to hold the lead in place. The lead, therefore, cannot be slipped through the anchor 100 along its longitudinal axis.

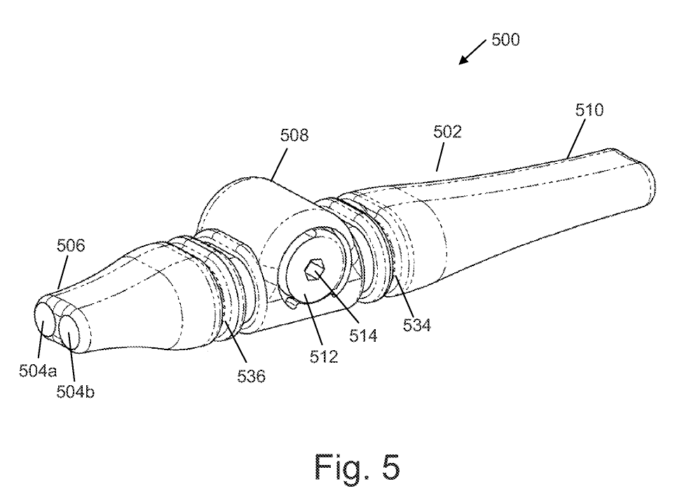

Referring now to FIG. 5, there is depicted an implantable anchor 500 according to an alternative embodiment. The anchor 500 includes a longitudinal body 502 with lumens 504a and 504b defined therein. In the illustrative embodiment, lumens 504a and 504b run longitudinally between ends 506 and 510, through middle portion 508. It is contemplated that the diameters of lumens 504a and 504b be of substantially equal measure, or could be varied to accommodate different sized leads.

As similarly shown in the embodiment illustrated in FIGS. 1-4, anchor 500 includes a rotatable cam 512 that extends laterally through anchor 500. Cam 512 includes an interface 514 for receiving a tool to facilitate the axial rotation of cam 512 in anchor 500. Cam 512 is similarly shaped as cam 112, and is operable in a first or open position and operable in a second or closed position (as similarly shown with cam 112 in FIGS. 3 and 4). Cam 512 is shaped such that that when cam 512 is placed in the first or open position, cam 512 does not inhibit lumens 504a and 504b such as to permit the insertion and movement of leads through lumens 504a and 504b. Cam 512 is further shaped such that when leads have been inserted through lumens 504a and 504b, and cam 512 is then placed in the second or closed position, portions of cam 512 extend into lumens 504a and 504b and engage each of the leads extending through lumens 504a and 504b and inhibit the movement of the leads with respect to anchor 500. It is contemplated that a locking mechanism, such as, but not limited to, a ratcheting mechanism, be utilized to further lock cam 512 in the second or closed position and further inhibit the rotation of cam 512 from the second position back towards the first position.

In use, cam 512 is place in the first or open position thereby permitting anchor 500 to be placed on the proximal ends of two leads and slid over the leads until the anchor is properly positioned along the leads. Cam 512 is then rotated to the second or closed position, such that cam 512 engages the leads extending through lumens 504a and 504b. Such engagement locks the anchor in place on the leads and inhibits movement relative thereto.

In certain embodiments, the anchor 500 may be fabricated using any suitable polymer processing technique. The polymer or polymers selected for the anchor 500 are preferably adapted for long term implantation. Biocompatibility and biostability are characteristics for the polymer selection for anchor 500. Also, the polymer preferably possesses a medium to high durometer to maintain the structural characteristics of anchor 500. An example of a suitable polymer for anchor 500 is polyetheretherketone (PEEK), although any biostable, biocompatible polymer having a suitable durometer and a suitable coefficient of friction can be employed.

In one embodiment, a combination of a relatively hard and soft (or flexible) material may be utilized. In this embodiment, the middle portion 508 of body 502 may be made from a relatively hard material, such as, but not limited to, select metals or PEEK. The end portions 506 and 510 may be fabricated from a more compliant material with a lower durometer value, such as silicone. Thus, the second end portions 506 and 510 are relatively flexible when compared to the middle portion 508 and may act as strain relief ports with respect to the middle portion 508.

In other embodiments, the longitudinal body 502 may be formed from a single piece of material. In such embodiments, the end portions may still function as strain relief ports. However, in these embodiments, the relative flexibility of the end portions may be due to geometric properties (such as thickness of the walls, etc.) and not from the different material properties of the component portions. Thus, the term flexibility as used herein may mean deformable (whether by choice of materials or geometry).

In some embodiments, the body 502 includes channels 534 and 536 which circumscribe body 502. Channels 534 and 536 are used to facilitate the suturing of anchor 500 to tissue of the patient.

The diameter of lumens 504a and 504b are sufficiently large to permit the introduction of leads with little difficulty. Thus, when cam 512 is in the open position, the anchor 500 may be freely moved along the leads (not shown). However, when the cam 512 is rotated to the second or dosed position, portions of cam 512 extend into lumens 504a and 504b, thereby decreasing the corresponding sizes of portions of lumen 504a and 504b smaller than the outer diameters of the leads. Thereby, upon rotation of cam 512 from an open to a dosed position, compressive forces are applied to the leads to cause anchor 500 to substantially hold the leads in place. The leads, therefore, cannot be slipped through the anchor 500 longitudinally.

Although the embodiment illustrated in FIG. 5 illustrates a single cam 512 that engages the leads in both lumens 504a and 504b, it is contemplated that two independently operated cams could be utilized, a cam corresponding to each of the lumens 504a and 504b.

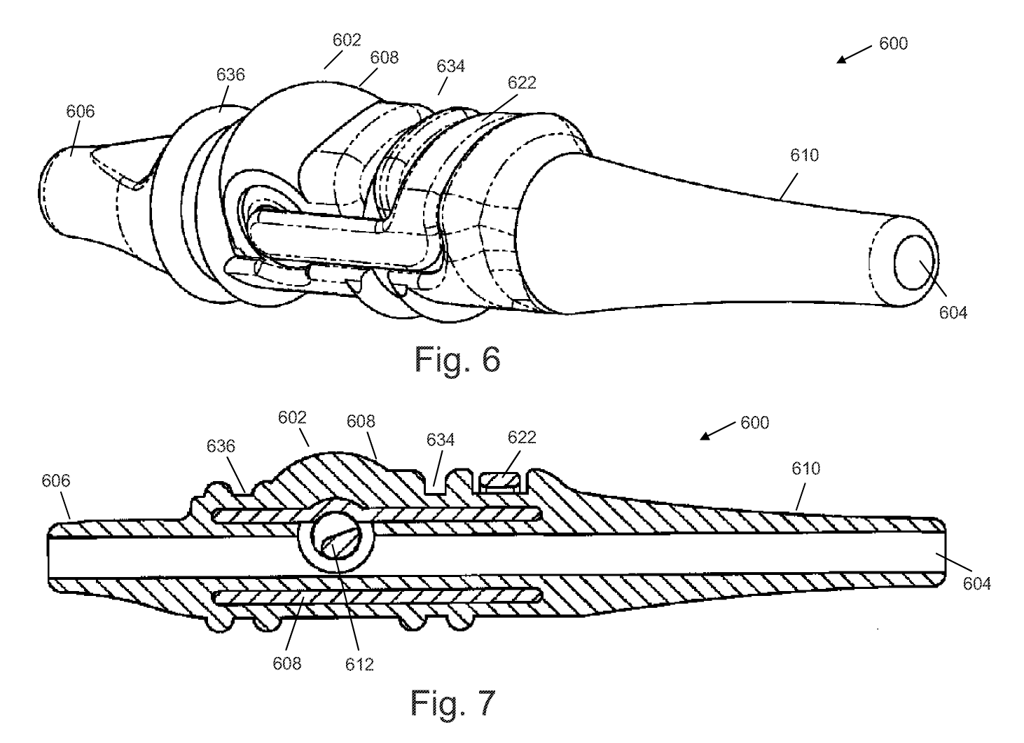

Referring now to FIGS. 6 and 7, there is depicted an implantable anchor 600 according to an exemplary alternative embodiment. The anchor 600 includes a longitudinal body 602 with a lumen 604 defined therein. In the illustrative embodiment, lumen 604 runs longitudinally between ends 606 and 610, through middle portion 608. Although depicted with a single lumen 604, it is contemplated that multiple lumens could be utilized, as similarly shown in FIG. 5 and described herein above.

As similarly shown in the embodiment illustrated in FIGS. 1-4, anchor 600 includes a rotatable cam 612 that extends laterally through anchor 600. A handle or arm 622 is integrated with cam 612, with the handle 622 for facilitating the axial rotation of cam 612 in anchor 600. Cam 612 is similar to cam 112, and is operable between a first or open position and a second or dosed position (as similarly shown with cam 112 in FIGS. 3 and 4), with FIG. 7 illustrating cam 612 in the dosed position. Cam 612 is shaped such that that when cam 612 is placed in the first or open position, cam 612 does not inhibit lumen 604 such as to permit the insertion and movement of a lead through lumen 604. Cam 612 is further shaped such that when a lead has been inserted through lumen 604, and cam 612 is then placed in the second or closed position, portions of cam 612 extend into lumen 604 and engage at least a portion of the lead extending through lumen 604 and inhibits the movement of the lead with respect to anchor 600. It is contemplated that a locking mechanism, such as, but not limited to, a ratcheting mechanism, be utilized to further lock cam 612 in the second or closed position and further inhibit the rotation of cam 612 from the second position back towards the first position.

In use, cam 612 is place in the first or open position by rotating arm 622, thereby permitting anchor 600 to be placed on the proximal end of a lead and slid over the lead until the anchor is properly positioned along the lead. Cam 612 is then placed in the second or closed position by rotating arm 622 to the position illustrated in FIGS. 6 and 7, such that cam 612 engages the lead extending through lumen 604. Such engagement locks the anchor in place on the lead and inhibits movement relative thereto.

In certain embodiments, the anchor 600 may be fabricated using any suitable polymer processing technique. The polymer or polymers selected for the anchor 600 are preferably adapted for long term implantation. Biocompatibility and biostability are characteristics for the polymer selection for anchor 600. Also, the polymer preferably possesses a medium to high durometer to maintain the structural characteristics of anchor 600. An example of a suitable polymer for anchor 600 is polyetheretherketone (PEEK), although any biostable, biocompatible polymer having a suitable durometer and a suitable coefficient of friction can be employed.

In one embodiment, a combination of a relatively hard and soft (or flexible) material may be utilized. In this embodiment, the internal portion 608 of body 602 and integrated therein, may be made from a relatively hard material, such as, but not limited to, select metals or PEEK and adds rigidity to the portion of anchor 600 to lock onto the lead. The remaining portions of anchor 600, including end portions 606 and 610 may be fabricated from a more compliant material with a lower durometer value, such as silicone. Thus, the portions 606 and 610 are relatively flexible when compared to the internal portion 608 and may act as strain relief ports.

In other embodiments, anchor 600 (less handle 622 and cam 612) may be formed from a single piece of material. In such embodiments, the end portions may still function as strain relief ports. However, in these embodiments, the relative flexibility of the end portions may be due to geometric properties (such as thickness of the walls, etc.) and not from the different material properties of the component portions. Thus, the term flexibility as used herein may mean deformable (whether by choice of materials or geometry).

In some embodiments, the body 602 includes channels 634 and 636 which circumscribe body 602. Channels 634 and 636 are used to facilitate the suturing of anchor 600 to the patient.

The diameter of lumen 604 is sufficiently large to permit the introduction of leads with little difficulty. Thus, when cam 612 is in the open position, the anchor 600 may be freely moved along the lead (not shown). However, when the cam 612 is in the second or dosed position, portions of cam 612 extend into lumen 604, thereby decreasing the corresponding size of the portion of lumen 604 smaller than the outer diameter of the lead. Thereby, upon rotation of arm 622 and cam 612 from an open to a dosed position, compressive forces are applied to the leads to cause anchor 600 to substantially hold the leads in place. The leads, therefore, cannot be slipped through the anchor 600 longitudinally.

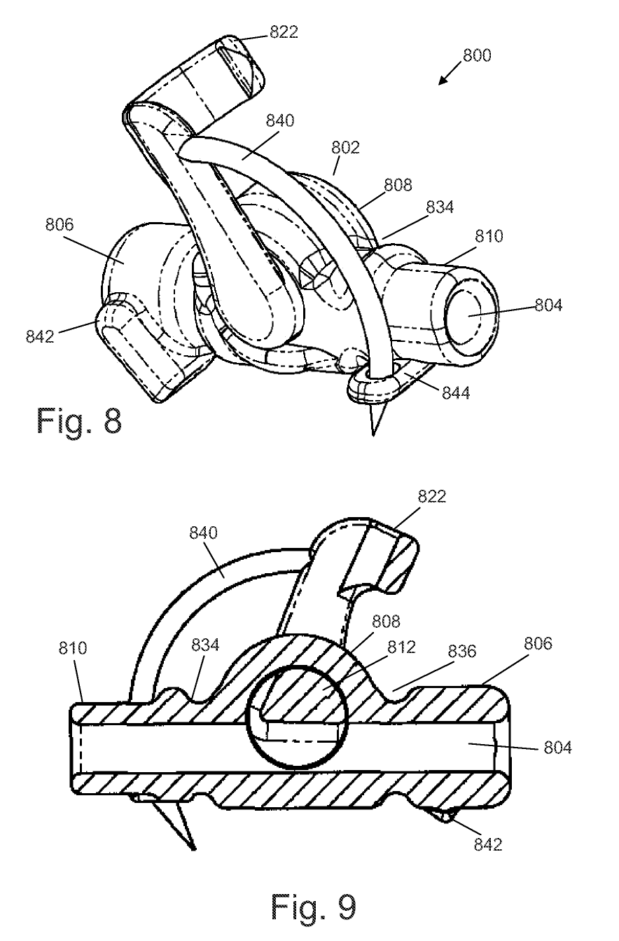

Referring now to FIGS. 8, 9, 10 and 11, there is depicted an implantable anchor 800 according to an exemplary alternative embodiment. The anchor 800 includes a longitudinal body 802 with a lumen 804 defined therein. In the illustrative embodiment, lumen 804 runs longitudinally between ends 806 and 810, through middle portion 808. Although depicted with a single lumen 804, it is contemplated that multiple lumens could be utilized, as similarly shown in FIG. 5 and described herein above.

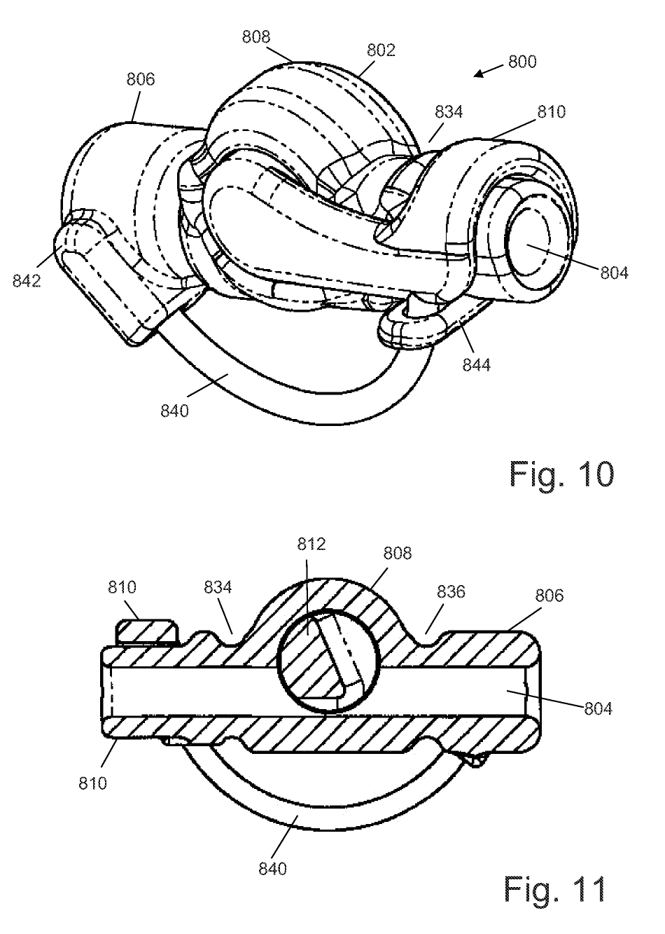

As similarly shown in the embodiments illustrated in FIGS. 1-4 and 6-7, anchor 800 includes a rotatable cam 812 that extends laterally through anchor 800. A handle or arm 822 is integrated with cam 812, with the handle 822 for facilitating the axial rotation of cam 812 in anchor 800. Cam 812 is similar to cams 112 and 512, and is operable between a first or open position (as depicted in FIGS. 8 and 9) and a second or closed position (as depicted in FIGS. 10 and 11). Cam 812 is shaped such that that when cam 812 is placed in the first or open position, cam 812 does not inhibit lumen 804 such as to permit the insertion and movement of a lead through lumen 804. Cam 812 is further shaped such that when a lead has been inserted through lumen 804, and cam 812 is then placed in the second or closed position, portions of cam 812 extend into lumen 804 and engage at least a portion of the lead extending through lumen 804 and inhibits the movement of the lead with respect to anchor 800. It is contemplated that a locking mechanism, such as, but not limited to, a ratcheting mechanism, be utilized to further lock cam 812 in the second or closed position and further inhibit the rotation of cam 812 from the second position back towards the first position.

Anchor 800 further includes a generally arcuate needle 840 which extends from handle 822. Needle 840 extends through anchor hole 844 and interfaces with a sleeve 842 when handle 822 and cam 812 are rotated to the closed position.

In use, cam 812 is place in the first or open position by rotating arm 822 (FIGS. 8 and 9), thereby permitting anchor 800 to be placed on the proximal end of a lead and slid over the lead until the anchor is properly positioned along the lead. Anchor 800 is then properly placed in a patient or user, whereby cam 812 is then placed in the second or closed position (FIGS. 10 and 11), such that cam 812 engages the lead extending through lumen 804. Such engagement locks the anchor in place on the lead and inhibits movement relative thereto. As cam 812 is placed in the second or closed position by rotating handle 822, needle 840 extends through tissue of the patient and into sleeve 842. This fixes or secures the anchor 800 in place with respect to the patient.

In certain embodiments, the anchor 800 may be fabricated using any suitable polymer processing technique. The polymer or polymers selected for the anchor 800 are preferably adapted for long term implantation. Biocompatibility and biostability are characteristics for the polymer selection for anchor 800. Also, the polymer preferably possesses a medium to high durometer to maintain the structural characteristics of anchor 800. An example of a suitable polymer for anchor 800 is polyetheretherketone (PEEK), although any biostable, biocompatible polymer having a suitable durometer and a suitable coefficient of friction can be employed. In one embodiment, anchor 800 is made from a relatively hard material, such as, but not limited to, select metals or PEEK and adds rigidity to anchor 600.

In some embodiments, the body 802 includes channels 834 and 836 which circumscribe body 802. Channels 834 and 836 may be used to facilitate additionally suturing of anchor 800 to the patient.

The diameter of lumen 804 is sufficiently large to permit the introduction of leads with little difficulty. Thus, when cam 812 is in the open position, the anchor 800 may be freely moved along the lead (not shown). However, when the cam 812 is in the second or closed position, portions of cam 812 extend into lumen 804, thereby decreasing the corresponding size of the portion of lumen 804 smaller than the outer diameter of the lead. Thereby, upon rotation of arm 822 and cam 812 from an open to a closed position, compressive forces are applied to the leads to cause anchor 800 to substantially hold the leads in place. The leads, therefore, cannot be slipped through the anchor 800 longitudinally.

Anchors according to representative embodiments may be utilized in conjunction with any suitable implantable medical device that comprises an implantable lead. For example, anchors 100, 500, 600 and 800 can be utilized to anchor a stimulation lead of a neurostimulation system as shown in FIG. 12, A neurostimulation system 1200 includes a pulse generator 1202 and one or more stimulation leads 1204. An example of a commercially available pulse generator is the EON® product available from Advanced Neuromodulation Systems, Inc. An example of a commercially available stimulation lead is the Axxess® lead available from Advanced Neuromodulation Systems, Inc.

The pulse generator 1202 is typically implemented using a metallic housing that encloses circuitry for generating the electrical pulses for application to neural tissue of the patient. The pulse generator 1202 is usually implanted within a subcutaneous pocket created under the skin by a physician. The lead 1204 is used to conduct the electrical pulses from the implant site of the pulse generator for application to the targeted nerve tissue via electrodes 1206. The lead 1204 typically includes a lead body of an insulative polymer material with embedded wire conductors extending through the lead body. The electrodes 1206 of the lead body are coupled to the conductors to deliver the electrical pulses to the nerve tissue. For example, the distal end 1208 of lead 1204 may be positioned within the epidural space of the patient to deliver electrical stimulation to spinal nerves to treat chronic pain of the patient. The anchors disclosed herein may be utilized to ensure that the distal end 1208 of the lead 1204 remains adjacent to the appropriate nerves associated with the chronic pain of the patient. In some embodiments, an “extension” lead (not shown) may be utilized as an intermediate connector if deemed appropriate by the physician.

In certain embodiments for SCS applications, the lead 1204 is a “body compliant” lead that possesses mechanical characteristics that allow the lead 1204 to stretch in response to forces experienced with the patient's body. For example, the lead 1204 may be adapted to stretch up to 25% in response to low stretching forces such as 2-2 pounds of force. The ability to exhibit significant elongation in response to such low forces enables the lead to be relatively robust (e.g., does not experience significant conductor breakage). Fabrication techniques and material characteristics for “body compliant” leads are disclosed in greater detail in U.S. Provisional Patent Application Ser. No. 60/788,518, entitled “Lead Body Manufacturing,” filed Mar. 31, 2006, which is incorporated herein by reference for all purposes.

Alternatively, the anchors 100, 500, 600 and 800 can be utilized to anchor an infusion catheter of an implantable drug infusion device 1300 as shown in FIG. 13. The implantable infusion drug pump device 1300 may include a central housing 1302, a reservoir 1304 to hold the infusate, a septum 1306 to allow infusate to be introduced into the reservoir, an energy source 1308 (e.g., a spring diaphragm) to drive the infusate from the reservoir and through an outlet port 1310, and various flow control elements (not shown).

The central housing 1302 of the device is often implanted in a suitable subcutaneous region with the septum 1306 positioned immediately below the skin of the patient to facilitate access to the reservoir 1304 for refilling purposes. A catheter 1312 is attached to the outlet port 1310 of the central housing 1302 to receive the infusate outflow. A distal end 1314 of the catheter is implanted within the patient adjacent to the appropriate therapy site. The anchors 100, 500, 600 and 800 may be utilized to ensure that the distal end 1314 of the lead 1312 remains adjacent to the appropriate site generating the chronic pain of the patient.

Although some representative embodiments have been discussed in terms of anchoring intrathecal and epidural catheters and leads, anchors can be employed according to alternative embodiments for any suitable location. For example, an anchor according to some embodiments could be used for peripheral nerve stimulation and gastric pacing applications.

Although representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure that processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

Any combination of the features discussed above are within the scope of certain embodiments of the present invention. Thus, a feature disclosed in reference to one embodiment may be combined with another embodiment. Furthermore, combinations of disclosed features and alternative features are within the scope of certain embodiments of the present invention.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

What is claimed is:

1. An implantable anchor for anchoring a medical lead within a user, the anchor comprising:
a body having a lumen, the lumen extending from a first end of the body to a second end of the body, the lumen configured to receive the medical lead;
a cam rotatably mounted within at least portion of the body intermediate the first end and the second end of the body, the cam rotatable between at least a first position and a second position, such that when the cam is rotated to the first position, a portion of the cam extends into the lumen, such that when the medical lead extends through the lumen, at least a portion of the cam engages the medical lead and substantially inhibits movement of the medical lead with respect to the anchor, and further when the cam is rotated to the second position, the cam is substantially free from the lumen, such that the movement of the medical lead is not substantially inhibited by the cam.

2. The implantable anchor of claim 1, wherein the cam includes an interface for receiving a tool to facilitate the rotation of the cam between the first position and the second position.

3. The implantable anchor of claim 1, further including a handle integrated with the cam, the handle for facilitating the rotation of the cam between the first position and the second position.

4. The implantable anchor of claim 3, further including a needle connected to the handle, the needle for securing the anchor to a patient.

5. The implantable anchor of claim 4, wherein the needle is generally arcuate in shape.

6. The implantable anchor of claim 1, further comprising a second lumen extending from the first end of the body to the second end of the body, the second lumen configured to receive a medical lead therein.

7. The implantable anchor of claim 6, wherein a second portion of the cam extends into the second lumen when the cam is placed in the first position.

8. The implantable anchor of claim 1, wherein the body includes a first strain relief portion, a second strain relief portion, and a center portion with the cam disposed within the center portion.

9. An implantable anchor for anchoring a medical lead within a living organism, the anchor comprising:
an elongated body having at least one lumen, the at least one lumen extending from a first end of the body to a second end of the body, the at least one lumen configured to receive the medical lead;
a cam mounted within at least portion of the body intermediate the first end and the second end of the body, the cam being rotatable between at least a first position and a second position, such that when the cam is rotated to the first position, a portion of the cam extends into the at least one lumen, such that when the medical lead extends through the lumen, at least a portion of the cam engages the medical lead and substantially inhibits movement of the medical lead with respect to the anchor, and further when the cam is rotated to the second position, the cam is substantially free from the lumen, such that the movement of the medical lead is not substantially inhibited by the cam.

10. The implantable anchor of claim 9, wherein the cam includes an interface for receiving a tool to facilitate the rotation of the cam between the first position and the second position.

11. The implantable anchor of claim 9, further including a handle integrated with the cam, the handle for facilitating the rotation of the cam between the first position and the second position.

12. The implantable anchor of claim 11, further including a needle connected to the handle, the needle for securing the anchor to a patient.

13. The implantable anchor of claim 12, wherein the needle is generally arcuate in shape.

14. The implantable anchor of claim 9, further comprising at least a second lumen extending from the first end of the body to the second end of the body, the at least second lumen configured to receive a medical lead therein.

15. The implantable anchor of claim 14, wherein a portion of the cam extends into the second lumen when the cam is placed in the first position.

16. An implantable anchor for anchoring a medical lead within a living organism, the anchor comprising:
an elongated body having at least one lumen, the at least one lumen extending from a first end of the body to a second end of the body, the at least one lumen configured to receive the medical lead;
the body including a first strain relief portion, a second strain relief portion, and a center portion;
a cam mounted within the center portion of the body, the cam being rotatable between at least a first position and a second position, such that when the cam is rotated to the first position, a portion of the cam extends into the at least one lumen, such that when the medical lead extends through the lumen, at least a portion of the cam engages the medical lead and substantially inhibits movement of the medical lead with respect to the anchor, and further when the cam is rotated to the second position, the cam is substantially free from the lumen, such that the movement of the medical lead is not substantially inhibited by the cam.

17. The implantable anchor of claim 16, wherein the cam includes an interface for receiving a tool to facilitate the rotation of the cam between the first position and the second position.

18. The implantable anchor of claim 17, wherein the cam includes an interface for receiving a tool to facilitate the rotation of the cam between the first position and the second position.

19. The implantable anchor of claim 17, further including a handle integrated with the cam, the handle for facilitating the rotation of the cam between the first position and the second position.

20. The implantable anchor of claim 19, further including a needle connected to the handle, the needle for securing the anchor to a patient, the needle being generally arcuate in shape.
